SEQUENCE LISTING (4 sequences)

SEQ ID NO: 1 - DNA, 18 bp, Artificial sequence
  telomerase substrate
aatccgtcgagcagagtt

SEQ ID NO: 2 - DNA, 24 bp, Artificial sequence
  telomerase primer
cccttacccttacccttaccctaa

SEQ ID NO: 3 - DNA, 24 bp, Artificial sequence
  telomerase primer
gcgcggctaaccctaaccctaacc

SEQ ID NO: 4 - DNA, 30 bp, Artificial sequence
  telomerase primer
gcgcggcttacccttacccttaccctaacc

(19) United States
(12) Patent Application Publication
(10) Pub. No.: US 2012/0070842 A1
(43) Pub. Date: Mar. 22, 2012

(54) Assay for Telomerase Activity Using Microfluidic Device

(76) Inventors: Calvin B. Harley, Murphys, CA (US); Hui Wang, Sunnyvale, CA (US); Zhu Zhen Pirot, Redwood City, CA (US)

(21) Appl. No.: 13/322,115

(22) PCT Filed: May 28, 2010
(86) PCT No.: PCT/US10/36719

§ 371 Date: Dec. 2, 2011

Related U.S. Application Data
(60) Provisional application No. 61/182,998, filed on Jun. 1, 2009.

(51) Int. Cl. C12Q 1/68

(52) U.S. Cl. 435/6.12

(57) ABSTRACT

Methods for determining the level of telomerase reverse transcriptase activity in mammalian cells are disclosed. A preferred measuring device is a microfluidic device that includes a spectrophotometer, a fluorescent detector, a fluorescence polarization detector or a scintillation counting device.

### REFERENCE TO PRIOR APPLICATIONS

This application claims the benefit of U.S. Provisional Application No. 61/182,998 filed Jun. 1, 2009, which is incorporated herein by reference in its entirety.

### FIELD OF THE INVENTION

The invention is directed to methods for determining the level of telomerase reverse transcriptase activity in cells using a microfluidic device.

### BACKGROUND

Telomeres are genetic elements located at the ends of all eukaryotic chromosomes which preserve genome stability and cell viability by preventing aberrant recombination and degradation of DNA (McClintock, 1941, Genetics vol 26, (2) pp 234-282; Muller, (1938) “The remaking of chromosomes’ The collecting net, vol 13, (8) pp 181-198). In humans, the telomeric sequence is composed of 10-20 kilobases of TTAGGG repeats (Blackburn, (1991) Nature vol. 350 pp 569-573; de Lange et al., (1990) Mol. Cell Biol. Vol 10, (2) pp 518-527). There is increasing evidence that gradual loss of telomeric repeat sequences may be a timing (“clock”) mechanism limiting the number of cellular divisions in normal cells (Allsopp et al., (1992) Proc. Natl. Acad. Sci. USA, vol. 89, pp. 10114-10118; Harley et al., (1990) Nature, vol. 345, pp. 458-460; Hastie et al., (1990) Nature, vol. 346, pp. 866-868; Vaziri et al., (1993) Amer. J. Hum. Genet., vol. 52, pp. 661-667). In contrast, immortal cells are capable of maintaining a stable telomere length by upregulating or reactivating telomerase, a ribonucleoprotein enzyme that is able to add TTAGGG repeats to the ends of chromosomes (Greider and Blackburn, (1985) Cell, vol. 43, pp. 405-413; Greider and Blackburn, (1989) Nature, vol. 337, pp. 331-337; Morin, (1989) Cell, vol. 59, pp. 521-529).

Methods for detecting telomerase activity, as well as for identifying compounds that regulate or affect telomerase activity, together with methods for therapy or diagnosis of cellular senescence and immortalization by controlling or measuring telomere length and telomerase activity, have been described. See PCT patent publication No. 93/23572, U.S. Pat. Nos. 5,629,154, 5,648,215, 5,645,986, 5,695,932 and 5,489,508. Each of the foregoing patent publications is incorporated herein by reference.

For example, U.S. Pat. Nos. 5,629,154; 5,863,726 and 5,648,215 describe in detail the preparation of a cell extract using a detergent lysis method and the analysis of telomerase activity by the Telomeric Repeat Amplification Protocol (TRAP assay). The telomerase activity assays described therein involve the extension of a nucleic acid substrate by telomerase and replication of extended substrates in a primer extension reaction, such as the polymerase chain reaction (PCR).

Other telomerase extraction methods use hypotonic swelling and physical disruption of cells and telomerase activity is assayed using an oligonucleotide substrate, a radioactive deoxyribonucleoside triphosphate (dNTP) for labelling any telomerase-extended substrate, and gel electrophoresis for resolution and display of products (Morin, (1989) Cell, vol. 59, pp. 521-529). Because telomerase stalls and can release the DNA after adding the first G in the 5′-TTAGGG-3′-telomeric repeat, the characteristic pattern of products on the gel is a six nucleotide ladder of extended oligonucleotide substrates. The phase of the repeats depends on the 3′-end sequence of the substrate; telomerase recognizes where the end is in the repeat and synthesizes accordingly to yield contiguous repeat sequences.

Using the Telomeric Repeat Amplification Protocol (TRAP assay), telomerase activity has been detected in 85% of primary human tumors tested from a variety of tissue types (Kim et al., (1994) Science, vol. 266, pp. 2011-2015; Shay and Bacchetti, (1997) European Journal of Cancer, vol. 33, No. 5, pp. 787-791). The detection of telomerase activity in human cells almost always correlates with indefinite proliferation capability (immortalization). U.S. Pat. No. 5,648,215 describes the presence of telomerase activity in somatic cells as indicative of the presence of immortal cells, such as certain types of cancer cells, which can be used to make that determination even when the cells would be classified as non-cancerous by pathology.

Previously the detection of the telomerase repeat amplification protocol (TRAP) products included polyacrylamide gel electrophoresis, ELISA, and QPCR or real-time PCR. There is a need for a higher-throughput, more accurate method of measuring the TRAP products.

### SUMMARY OF THE INVENTION

The invention provides a method for measuring telomerase activity in a cell, cell extract or subject comprising measuring the level of telomerase activity in the cell or subject.

Conventional read out method for telomerase repeat amplification protocol (TRAP) products include polyacrylamide gel electrophoresis, ELISA and QPCR or real-time PCR. Here we describe a new method for the TRAP measurement, e.g. measuring TRAP DNA ladders with a microfluidics system.

In one embodiment the invention provides a method of measuring telomerase repeat amplification products in a cell or tissue reaction mixture comprising flowing the telomerase repeat amplification products in a channel of a microfluidic device and measuring the amount of telomerase repeat amplification products in the reaction mixture.

In another embodiment, the invention provides a method of measuring telomerase activity in a cell or tissue extract, the method comprising the steps of:

(a) placing an aliquot of cell or tissue extract in a reaction mixture comprising a telomerase substrate and a buffer in which telomerase can catalyze extension of said telomerase substrate by addition of telomeric repeat sequences to generate telomeric extension products which are then amplified to generate telomerase repeat amplification products;
(b) separating the telomerase repeat amplification products in a channel of a microfluidic device, and
(c) measuring the amount of telomerase repeat amplification products in the reaction mixture.

In another embodiment, the amplification step of the method comprises adding to said reaction mixture a primer comprising a sequence sufficiently complementary to a telomeric repeat to hybridize specifically thereto under conditions such that if a telomeric extension product is present in said reaction mixture, said primer will hybridize to said telomeric extension product and extend to form a complementary copy of said telomeric extension product, thereby forming telomerase repeat amplification products.

The method may further comprise correlating the presence of telomerase activity in said cell extract with the presence of telomerase repeat amplification products and absence of telomerase activity in said cell sample with absence of said telomerase repeat amplification products.

In another embodiment, the invention is directed to a method for evaluating the biological response of a subject exposed to a telomerase modulator comprising obtaining a cell or tissue sample from the subject, generating a telomerase repeat amplification product reaction mixture from the subject's cell or tissue sample, separating the telomerase repeat amplification products in a channel of a microfluidic device and measuring the amount of telomerase repeat amplification products in the reaction mixture.

In another embodiment, the invention is directed to a method for evaluating the biological response of a subject exposed to a telomerase modulator comprising the steps of:

(a) placing an aliquot of cell or tissue extract from the subject after exposure to the telomerase modulator in a reaction mixture comprising a telomerase substrate and a buffer in which telomerase can catalyze extension of said telomerase substrate by addition of telomeric repeat sequences to generate telomeric extension products which are then amplified to generate telomerase repeat amplification products;
(b) separating the telomerase repeat amplification products in a channel of a microfluidic device,
(c) measuring the amount of telomerase repeat amplification products in the reaction mixture, and
(c) correlating the level of telomerase activity in said cell extract with the level of said extended telomerase substrate.

It is contemplated that the level of telomerase activity in the cell extract of the subject will be compared to the level of telomerase activity in a cell extract of the subject prior to treatment with the telomerase modulator. Alternatively the level of activity might be compared to a standard level of telomerase activity for the type of cell extract over a population of subjects. A biological response would be an increase or decrease in the level of telomerase activity.

In other embodiments, the amplification step of the method comprises adding to said reaction mixture a primer comprising a sequence sufficiently complementary to a telomeric repeat to hybridize specifically thereto under conditions such that if a telomeric extension product is present in said reaction mixture, said primer will hybridize to said telomeric extension product and extend to form a complementary copy of said telomeric extension product, thereby forming telomerase repeat amplification products.

The amplification step of the invention may further comprise heating said reaction mixture to denature said telomerase repeat amplification products; and cooling said reaction mixture to a temperature at which complementary nucleic acids can hybridize and said primer can extend if extended telomerase substrates are present.

The amplification step may have template-dependent DNA polymerase present in the reaction mixture and said primer may extended by addition of nucleotides to said primer by said DNA polymerase. The template-dependent DNA polymerase is a thermostable template-dependent DNA polymerase.

In some embodiments, the telomerase substrate may be labeled. In some embodiments the primer may be labeled. Where there is a label, it may be selected from the group consisting of a radioactive molecule, a fluorescent molecule, a phosphorescent molecule, a ligand for a receptor, biotin, and avidin.

In some embodiments the telomerase repeat amplification products are duplex DNA which may be labeled with an intercalating label selected from the group consisting of a radioactive molecule or a fluorescent molecule.

In some embodiments the telomerase substrate lacking a telomeric repeat sequence is 5′-AATCCGTCGAGCAGAGTT-3′ (SEQ ID NO:1). In some embodiments, the primer comprises a non-telomeric repeat sequence at a 5′-end of said primer. In some embodiments the primer is 5′-CCCTTACCCTTACCCTTACCCTAA-3′ (SEQ ID NO: 2), 5′-GCGCGGCTAACCCTAACCCTAACC-3′ (SEQ ID NO:3) or 5′-GCGCGGCTTACCCTTACCCTTACCCTAACC-3′ (SEQ ID NO:4).

In some embodiments, the method of measuring the telomerase activity further comprises normalizing the level of telomerase activity in the cell extract relative to the amount of RNA or protein in the cell extract. In some embodiments the amount of protein is the total amount of protein in the cell extract. In some embodiments, the amount of RNA in the cell extract is the amount of ribosomal RNA. The amount of ribosomal RNA may be determined by a PCR reaction using primers for the 18S ribosomal RNA. In other embodiments, the amount of RNA in the cell extract may be the amount of mRNA of genes which are specifically expressed in follicle cells.

In one embodiment the subject is a mammal. The mammal is selected from domesticated mammals such as dogs, cats, horses, mice, rats etc. In some embodiments, the mammal is a human.

The cells selected are cancer cells, skin cells, hair follicle cells, blood cells. The cancer cells are selected from the group consisting of breast cancer, ovarian cancer, basal-cell carcinoma, small-cell lung carcinoma, non-small cell lung carcinoma, squamous cell carcinoma, hepatocellular carcinoma, renal cell carcinoma, and multiple myeloma.

In some embodiments, the microfluidic device comprises a detector selected from the group comprising a spectrophotometer, a fluorescent detector, a fluorescence polarization detector or a scintillation counting device.

In some embodiments the detector is coupled to a computer which computer comprises instructions that convert a signal from the detector into the level and/or size of the telomerase repeat amplification products generated by the reaction mixture.

These and other objects and features of the invention will become more fully apparent when the following detailed description of the invention is read in conjunction with the accompanying drawings.

### BRIEF DESCRIPTION OF THE DRAWINGS

FIG. 1 is a picture of the microfluidic device readout for different concentrations of the TRAP reaction mixture.

FIG. 2 shows three graphs comparing the sensitivity of detection by increasing the number of PCR cycles in the TRAP assay.

FIG. 3A is a picture of an acrylamide gel. FIG. 3B is the readout of the microfluidic device from the same TRAP assay. FIG. 3C is a comparison of the relative telomerase activity of the analysis of the TRAP reaction using the acrylamide gel versus the microfluidic device over a range of different concentrations of hair follicle extract.

FIG. 4 A is a picture of an acrylamide gel and bar graph and FIG. 4 B is the readout of the microfluidic device and bar graph both from the same TRAP reaction after treatment of the hair follicle extract with different concentrations of Imetelstat (GRN163L).

FIG. 4C is a graph showing the inhibition of telomerase activity with different concentrations of Imetelstat.

FIG. 5A is a picture of an acrylamide gel and bar graph and FIG. 5B is the readout of the microfluidic device and bar graph both from the same TRAP reaction after treatment of the A549 cell lysate with different concentrations of Imetelstat (GRN163L).

FIG. 6A is a picture of an acrylamide gel and bar graph and FIG. 6B is the readout of the microfluidic device and bar graph both from the same TRAP reaction after treatment of the HEK293 cell lysate with different concentrations of Imetelstat (GRN163L)

### DETAILED DESCRIPTION OF THE INVENTION

### I. Definitions

The terms below have the following meanings unless indicated otherwise.

A “hair follicle” refers to the plucked hair of a subject which includes the hair follicle at the base of the hair, including the bulb and the sheath. In rodents, cats, rabbits and dogs the hair is preferably the whisker or vibrissa hair.

A ‘subject” refers to a mammal. The cell or tissue extract may be obtained from mammals such as humans; agriculturally important mammals, such as cattle, horses, sheep; and/or veterinary mammals, such as cats, rabbits, rodents and dogs.

A “cell extract” refers to the biological extract obtained from cells. The cells may be cells from any organism which is known to have telomerase activity. Such organisms include eukaryotic organisms such as Tetrahymena, yeast and mammalian organisms. In mammals, such cells may be selected from hair follicle cells, peripheral blood cells, cancer cells, buccal cells, skin cells or any other cells from the subject.

A “primer comprising a sequence sufficiently complementary to a telomeric repeat” includes a primer that may contain one or more mismatched bases within the repeats which are complementary to the telomerase substrate extension product to which the primer is intended to hybridize. The number of mismatches that can be tolerated within this definition can vary depending upon the length and sequence composition of the primer, the temperature and reaction conditions employed during the PCR step. A “CX primer” is also called a “reverse primer” is composed of sequences complementary to imperfect telomeric repeats and one perfect repeat. For example the primer may be 5′-(CCCTTA)₃CCCTAA-3′ (SEQ ID NO:2).

The ‘telomerase substrate” or “TS” is an oligonucleotide chosen to be recognized by the telomerase to be tested. If one is using the present method to determine the level of telomerase activity in a human subject, one employs a telomerase substrate recognized by human telomerase. Preferably when one employs a DNA polymerase based primer extension step, the telomerase substrate should not comprise a complete telomeric repeat sequence to minimize primer dimer formation. For instance, a human telomerase substrate of the invention is oligonucleotide TS, which contains a sequence at its 3′ end that is identical to five of the six bases of the human telomeric repeat but otherwise contains no complete telomeric repeat sequences.

The “intercalating label” is a label or dye which can tightly bind to double stranded nucleic acid. Preferably the label is a fluorescent label such as cybergreen or cybergold (Molecular Diagnostics/Biomerieux, Etoile, France).

The Primer label is a label or dye which can be covalently bound to a nucleic acid. Examples of such a label are fluorescein, Cy 3 or C3 (GE Healthcare Life Sciences, Piscataway, N.J.). Examples of a radioactive label are ³²P or ³³P.

“Telomerase activity” is the activity of a telomerase reverse transcriptase. In particular, the activity of the telomerase is the addition of telomeric DNA repeats to a telomerase substrate per unit time. In particular the telomerase activity is the level of activity in a mammalian cell or tissue and in particular in a human cell or tissue.

A “telomerase modulator” is a compound that directly or indirectly either inhibits or activates the expression or activity of telomerase. A “telomerase modulator” may be a “telomerase inhibitor” or a “telomerase activator”.

A “telomerase inhibitor” is a compound that directly or indirectly inhibits or blocks the expression or activity of telomerase. A telomerase inhibitor is said to inhibit or block telomerase if the activity of the telomerase in the presence of the compound is less than that observed in the absence of the compound. Preferably the telomerase is human telomerase. More preferably, the telomerase inhibitor is an hTR template inhibitor. An “hTR template inhibitor” is a compound that blocks the template region (the region spanning nucleotides 30-67 of SEQ ID NO: 1 herein) of the RNA component of human telomerase, thereby inhibiting the activity of the enzyme. For example, a telomerase inhibitor is GRN163L. (See U.S. Pat. No. 7,494,982 which is incorporated by reference herein)

A “cancer” is a malignant tumor. In particular, the cancer is a malignant tumor of epithelial-cell origin, that is, a malignant tumor that begins in the lining layer (epithelial cells) of organs. At least 80% of all cancers are carcinomas, and include breast cancer, both ductal and lobular carcinomas of the breast; ovarian cancer; basal-cell carcinoma, the most common non-melanoma skin cancer; squamous cell carcinoma, a common form of skin cancer and the most common type of lung cancer; hepatocellular carcinoma, the most common form of liver cancer; renal cell carcinoma, a malignant tumor located of the kidneys; and transitional cell carcinoma, a type of cancer that develops in the lining of the bladder, ureter, or renal pelvis. The cancer cells making up a carcinoma are referred to as “carcinoma cells.” Also includes in the term “cancer” are cancers of the blood cells such as leukemias, lymphomas and myelomas.

A “microfluidic device” or “microfluidic system” is a microfluidic chip which has a network of microscale cavities (channels or chamber etc.) having a least one dimension less than about 1 millimeter through which fluids or chemicals are moved. See for example, U.S. Pat. No. 6,042,709 and U.S. Pat. No. 6,287,520 and U.S. Patent Application No. 2008/0090244. Commercial systems that are available include the LabChip HT DNA from Caliper Technologies (Mountain View, Calif.).

All articles, books or journals referenced herein are incorporated herein in their entirety.

### II. Measurement of Activity

The present invention provides novel methods for the detection of telomerase activity in a subject or cell extracts from the subject.

The TRAP assay is a standard method for measuring telomerase activity in a cell extract system (Kim et al., Science 266:2011, 1997; Weinrich et al., Nature Genetics 17:498, 1997). Briefly, this assay measures the amount of nucleotides incorporated into elongation products (polynucleotides) formed by nucleotide addition to a labeled telomerase substrate or primer. This method is described in detail in U.S. Pat. Nos. 5,837,453, 5,863,726 and 5,804,380, as well as in U.S. Pat. Nos. 5,629,154 and 5,648,215, which are incorporated herein in their entirety. The use of the TRAP assay in testing the activity of telomerase inhibitory compounds is described in various publications, including WO 01/18015. In addition, the following kits are available commercially for research purposes for measuring telomerase activity: TRAPeze™ XK Telomerase Detection Kit (Millipore; Billerica Mass.) and TeloTAGGG Telomerase PCR ELISA plus (Roche Diagnostics, Indianapolis Ind.).

To practice the TRAP method, one first prepares a cell extract; preferably using a detergent based extraction method and then places the cell extract or an aliquot of the cell extract in a reaction mixture comprising a telomerase subtract and a buffer compatible with telomerase activity. The particular telomerase substrate chosen may vary depending on the type or origin of the telomerase activity for which one is testing. The telomerase activity expressed by one mammal may differ with respect to substrate specificity from that expressed by another mammal. Consequently, if one is using the method to determine the effect of a telomerase modulator on a human, one employs a telomerase substrate recognized by human telomerase.

When one employs a DNA polymerase-based primer extension step, the present method requires that the telomerase substrate not comprise a telomeric repeat sequence. The human telomerase adds repeats of sequence 5′-TTAGGG-3′. Thus, if one is using the present method to assay for human telomerase activity, the telomerase substrate should be a human telomerase substrate lacking the sequence 5′-TTAGGG-3′.

This requirement for the telomerase substrate to lack telomeric repeat sequences arises out of the second reaction of the present method, the non-telomerase-mediated primer extension reaction. In this reaction, an oligonucleotide primer that hybridizes only to extended telomerase substrates is added to the reaction mixture under conditions such that, if extended telomerase substrates are present, the primer binds to the extended substrates and is then extended by enzymatic action. Because telomerase can extend the telomerase substrate only by the addition of telomeric repeats, the oligonucleotide primer will necessarily comprise a sequence complementary to a telomeric repeat. If the telomerase substrate sequence employed in the telomerase extension reaction comprised a telomeric repeat, then the primer employed in the primer extension reaction could hybridize to unextended telomerase substrate, resulting in false positive results.

The primer extension reaction conducted subsequent to the telomerase substrate extension serves to amplify the signal produced by the presence of telomerase activity in a sample (extended telomerase substrates) by producing a second signal (extended primers). The primers can be extended by any means that requires the presence of extended telomerase substrates for primer extension to occur; preferred means are mediated by a template-dependent DNA or RNA polymerase, a template-dependent DNA ligase, or a combination of the two. With these means, if telomerase activity is present in the sample, an extended telomerase substrate is formed and then hybridizes to a primer, providing a substrate for either DNA or RNA polymerase or DNA ligase to produce a primer extension product.

Once a primer extension product has formed, one can disassociate (typically by heating, but one could also use an enzyme or chemical process, such as treatment with helicase) the extended primer from the extended substrate. If additional primer and primer extension reagent is present in the sample, then a new primer/extended telomerase substrate complex can form, leading to the production of another extended primer. One can repeat the process of primer extension and denaturation several to many times, depending upon the amount of signal desired. Typically, primer extension and denaturation of extended primer/extended telomerase substrate complexes will be performed at least 5, 10, 15, 20, 25 to 45 or more times, from 20 to 38 times, from 25 to 35 times. Moreover, if a second primer complementary to the 3′-end of the extended primer is present in the reaction mixture, one can increase the signal (both extended primer and also additional extended telomerase substrate) dramatically. Unextended telomerase substrate still present in the reaction mixture during the primer extension step can function as such a second primer.

Those of skill in the art will recognize that if the primer extension reagent is a DNA polymerase, and a second primer is present, one has the requisite components for a polymerase chain reaction, more fully described in U.S. Pat. Nos. 4,683,195; 4,683,202; and 4,965,188, provided the appropriate buffer and nucleoside triphosphates are present in the reaction mixture. PCR amplification is a preferred mode for conducting the primer extension reaction step of the present invention and dramatically increases sensitivity, speed, and efficiency of detecting telomerase activity as compared to the conventional assay.

The telomerase assay is particularly well suited for providing a variety of means to quantitate the amount of telomerase in a sample.

PCR normalization of the intensity of the telomerase ladder to that of the internal standard permits the assay to become linear so that accurate comparisons between samples can be made, as is described in the Examples section below. A weak signal resulting from the internal standard relative to that in other samples could indicate limiting PCR conditions, thus allowing the practitioner to choose to repeat the assay under non-limiting conditions, for example, by providing higher polymerase levels. The inclusion of the internal standard also immediately identifies potentially false negative samples.

One means for obtaining quantitative information is the use of a PCR control oligonucleotide template added to each reaction mixture in a known amount. An illustrative PCR control oligonucleotide comprises, in 5′-3′ order, a telomerase substrate sequence, a spacer sequence (which can be any sequence of nucleotides or length and can alter spacing of the ladder produced by electrophoresis of reaction products produced from telomerase containing samples), a telomeric repeat sequence (typically present in multiple, i.e., 2 to 50, copies), and a sequence complementary to the primer used in the assay (and so which may simply be a portion of the telomeric repeat sequence). Of course, an oligonucleotide complementary to the control sequence defined above can also serve as the control sequence, and a double-stranded control nucleic acid can also be employed.

Alternatively, one can add a PCR control nucleic acid of any sequence to the reaction mixture in known amounts and amplify the control with primers which can be the same as or different from those used to amplify the extended telomerase substrate. The control oligonucleotide and/or the primers used to amplify the control oligonucleotide can be labelled identically to or differently from the label used to detect the telomerase extension products. Use of an internal control not only facilitates the determination of whether the assay was conducted properly but also facilitates quantitation of the telomerase activity present in the sample. The detailed protocol for conducting TRAP assays using primer and internal control is described in U.S. Pat. Nos. 5,629,154, and 5,863,726 which are incorporated herein in their entirety.

Moreover, a variety of different types of oligonucleotides can be used in telomerase activity assays. While the discussion above and Examples below illustrate assay methods with results obtained using oligodeoxyribonucleotide telomerase substrates and primers with DNA polymerase, the activity assay used in the present invention is not so limited. Thus, one can employ oligoribonucleotides or oligonucleotides that comprise one or more modified (i.e., synthetic or non-naturally occurring) nucleotides in the telomerase assay. In similar fashion, one can employ an RNA polymerase to extend a primer or to copy an extended telomerase substrate. These and other variations of the present method will be apparent to those of skill in the art upon consideration of this description of the invention.

The intensity of the telomerase product generated may also be normalized relative to a control molecule such as, for example, RNA or total protein so that comparisons between samples can be made. This provides correction for the extraction efficiency of telomerase from the cellular extract allowing different samples to be compared. The activity of the telomerase is expressed as a value relative to protein amount or RNA amount. In particular, the amount of total protein or the amount of RNA can be used for normalization purposes. Where ribosomal RNA serves as the normalization control, the ribosomal RNA can be determined by a PCR reaction using primers directed to the 18S ribosomal RNA. Alternatively the amount of mRNA in the cell extract may be determined by measuring the mRNA for genes specifically expressed in the cells or interest or housekeeping genes.

The level of activity measured by the telomerase assay after exposure to a telomerase modulator can be compared to the telomerase activity prior to exposure to the telomerase modulator. A difference in activity is observed when there is at least a 50% increase, at least a 2 fold increase, at least a 4 fold increase or at least a 6 fold increase in activity after exposure to a telomerase activator. A difference is activity is observed when there is less than 90% of the activity, less than 80% of the activity, less than 70% of the activity or less than 50% of the activity after exposure to a telomerase inhibitor.

The invention provides new ways of determining how many telomeric repeats are added to the telomere substrate and how many copies of each number of telomeric repeats are present in the reaction mixture by considering how much signal from the different sized telomerase repeat amplification products are present in the reaction mixture and comparing those levels to the expected level.

A microfluidic device comprising a channel or chamber is configured to allow the separation of different sized telomerase repeat amplification products in one or more aliquots. A detector integral with or proximal to the microfluidic device is configured to detect a reproducible shape, length, width, volume or area occupied by the amplified copies of the nucleic acid of interest present in one of the aliquots following separation of the nucleic acid in the aliquots.

The detector is typically configured to detect one or more electromagnetic energy signal in or on the microfluidic device, although other device sensors e.g. pH, conductivity, radioactivity etc) can also be used. For example, the detector can detect fluorescence, luminescence and or fluorescence polarization of the sample. See for example U.S. Pat. No. 7,238,323 and U.S. Patent Application No. 2008/0090244 which are incorporated herein by reference.

The method may also include software with instructions for correlating the shape, length, width volume or area occupied by amplified copies of the nucleic acid of interest present in one of the aliquots to the number of copies of the nucleic acid of interest present in the sample. The computer software can also include statistical analysis of the signals received from one or more of the aliquots. The statistical analysis optionally comprises quantitatively determining a concentration, proportion or number of the nucleic acids of interest in the sample. See for example U.S. Pat. No. 7,238,323 and U.S. Patent Application No. 2008/0090244 which are incorporated herein by reference.

The following examples are offered by way of illustration and not by way of limitation.

### EXAMPLES

### Example 1

### Measuring Telomerase (TRAP) Activity by Microfluidic Device

This procedure was used to assess telomerase activity in hair follicles, cultured cells (tumor or immortalized) and tissue samples. The assay assesses in vitro telomerase activity by measuring the extension of an oligonucleotide substrate by telomerase present in test samples, followed by PCR amplification of the extended DNA called Telomeric Repeat Amplification Protocol (TRAP). In this example the method of measurement for the TRAP activity, e.g. measuring TRAP DNA ladders by LabChip® GX microfluidics system was compared to measurement with sequencing gel. The telomerase products incorporate Cy5, which is attached to the 5′ end of the telomerase substrate (TS). Cy5-TS also acts as the forward primer and is integrated in the PCR products, which are fluorescent at 635/700 nm and detected by the microfluidics system. It was found that the total signal of specific DNA fragment peaks was proportional to the telomerase activity from the test samples.

Human embryonic kidney (HEK293) cells and human hair follicle extracts were prepared by adding M-Per buffer (Pierce Thermo Fisher Scientific, Rockford Ill.) to lyse the cells for 1-6 hr, then the lysate was centrifuged at 14000 rpm in an Eppendorf Centrifuge 5417R (Westbury N.Y.) for 20 min at 4° C. The supernatant was transferred into a new tube.

| 10× TRAP buffer: | |
|---|---|
| Tris-HCl pH 8.3 | 200 mM |
| MgCl2 | 15 mM |
| KCl | 650 mM |
| Tween 20 | 0.5% |
| EGTA | 10 mM |
| BSA | 1 mg/ml |

| Primers: | |
|---|---|
| (SEQ ID NO: 1) | |
| Cy5-TS | (AAT CCG TCG AGC AGA GTT) 5′ |
| (SEQ ID NO: 4) | |
| ACX | (GCGCGGCTTACCCTTACCCTTACCCTAACC) |

Taq polymerase is AmpliTaq DNA Polymerase, Applied Biosystems, cat. # N8080171 and dNTP from Invitrogen, cat. # R72501

### TRAP Assay Set Up

| | | μL, per | | | |
|---|---|---|---|---|---|
| | Stock Conc. | rxn | | Final Conc. | |
| 10x TRAP buffer | | 5 | | 1x | |
| dNTP | 2.5 mM | 1 | | 50 | uM |
| Cy5-TS | 0.5 mg/ml, 83 uM | 0.1 | | 1 | ng/μL |
| ACX | 0.1 mg/ml, 11 uM | 1 | | 2 | ng/μL |
| Taq polymerase | 5 U/μL | 0.4 | | 0.04 | U/μL |
| H2O | | 37.5 | | | |
| total | | 45 | | | |
| Cell/tissue | | 5 | μl | | |
| extract | | | | | |

5-10 μL of extract per reaction was used in the TRAP reaction adjusting the volume with H₂O.

The telomere extension and PCR amplification was conducted as follows: 30° C. for 30 minutes

a. 28 to 33 cycles of the following 3-step reaction:
94° C. for 30 seconds
60° C. for 30 seconds
72° C. for 1 minute
   b. 72° C. for 4 minutes
   c. Hold at 4° C.

The TRAP reaction products were then either run on an acrylamide gel or run in the LABCHIP® GX microfluidics system (Caliper Life Sciences, Mountain View, Calif.) as follows. The Labchip®GX/GXII microfluidics system has a size range of 25-1000 bp, size resolution of 15% from 25-100 bp, sizing recovery of 10% sizing precision of 5% CV, linear concentration range of 0.1 ng/μL-50 ng/μL per fragment, and sensitivity of 0.1 ng/μL. 400 samples can be measured per chip.

The Gel solution was prepared by adding 12.5 μL of DYE CONCENTRATE™ (Caliper Life Sciences, Mountain View, Calif.) to 1.0 mL of GEL MATRIX™ (Caliper Life Sciences) by reverse pipetting technique and then clarifying by placing the mixture in tubes having spin filters (500 μL each) and centrifuging at 9000 rcf for 8 minutes at room temperature.

The microfluidic device was loaded with the established LADDER SOLUTION™ having 10 μL of DNA ladders (Caliper Life Sciences, Mountain View, Calif.) in 90 μL of 1× TRAP reaction buffer without BSA. The microfluidic device was loaded with Buffer which is 1× TRAP reaction buffer without BSA. 25 μL of the TRAP reactions were loaded onto a 96-well skirted V-shaped plate (BioRad, Hercules Calif.) and that was placed into the microfluidic machine.

The DNA chip was prepared according to the manufacturer's instructions by injecting the of Dye/Gel mixture into the microfluidic chip using reverse pipetting technique. 50 μL of DNA MARKER™ (Caliper Life Sciences, Mountain View, Calif.) dye markers was added to the chip. The chip was placed in the machine and run according to the manufacturer's instructions.

Using LABCHIP GX® software (Caliper Life Sciences, Mountain View, Calif.) the minimum peak height on the “Peak Find” tab was adjusted to an appropriate number to identify all TRAP product peaks. In this case the minimum peak find was set at 1. The amount of DNA in each peak in the reaction mixture was quantified relative to the known concentration of DNA in the peaks in the DNA ladder. The total amount of telomerase repeat amplification product was determined by adding the total peak fluorescent level over all of the peaks and subtracting the fluorescence of the primer peak. The total double strand DNA fragment concentration (ng/mL) from each reaction was the measurement of telomerase activity of each testing sample.

In parallel, 25 μL of each TRAP reaction was loaded onto a polyacrylamide gel (19% acrylamide/1% N,N′-methylene-bis acrylamide)(BioRad Lab, Inc. Hercules Calif.). The intensity of the bands on the gel was measured and compared to the results obtained with the microfluidic device.

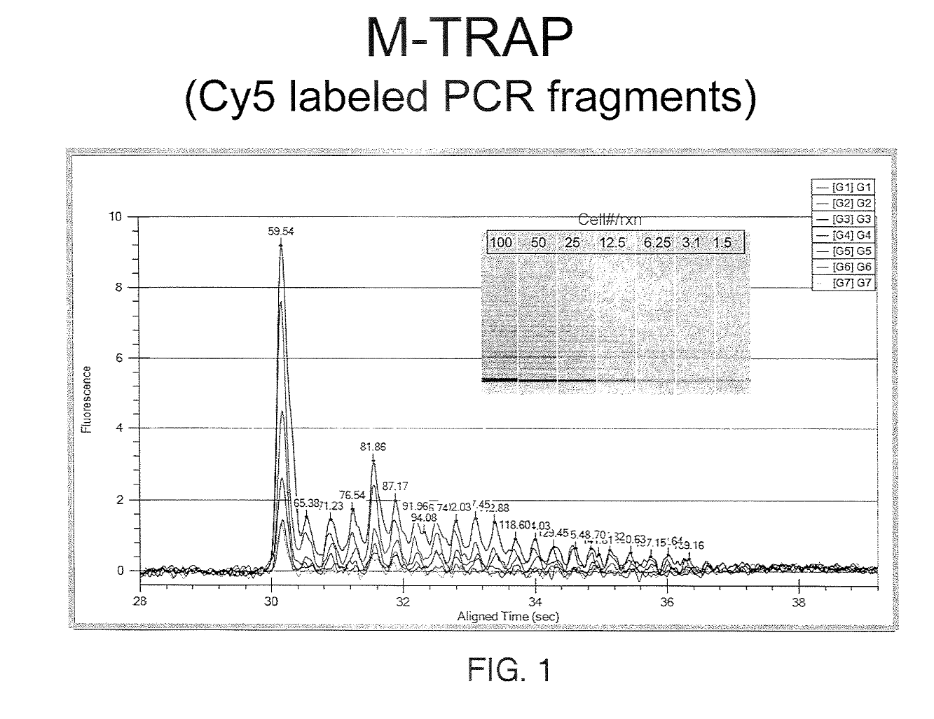

FIG. 1 is a picture of the microfluidic device readout for different concentrations of the TRAP reaction mixture from HEK293 cell lysate. It was found that the microfluidic readout was comparable to the results from the acrylamide gel.

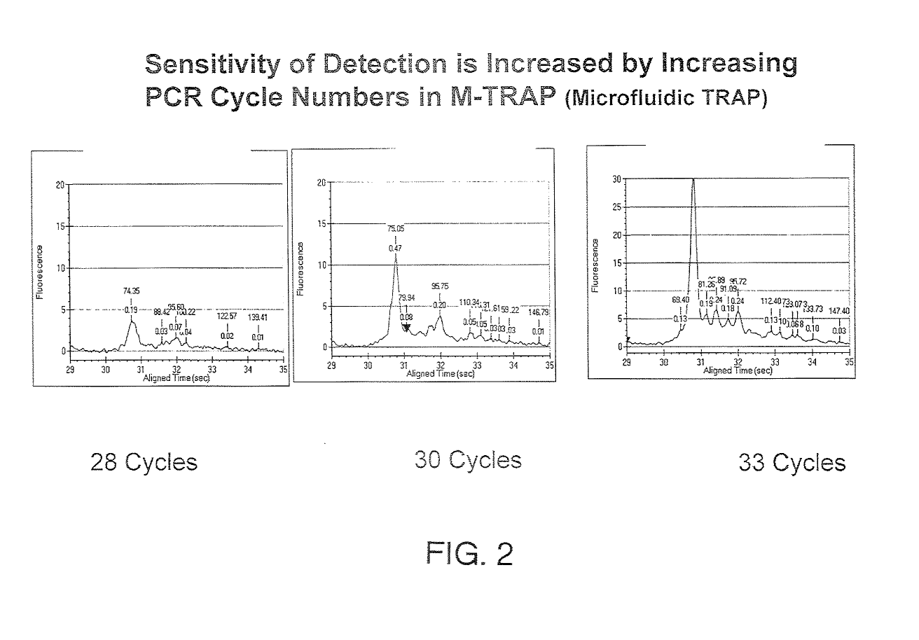

FIG. 2 shows three graphs comparing the sensitivity of detection by increasing the number of PCR cycles in the TRAP assay from human hair follicle cell extracts for 28, 30 or 33 PCR cycles. 5 μL, of hair follicle extract ( 1/12 of hair follicle) was assayed per reaction. It was found that increasing the number of cycles from 28 cycles to 33 cycles improved the resolution in the microfluidic readout.

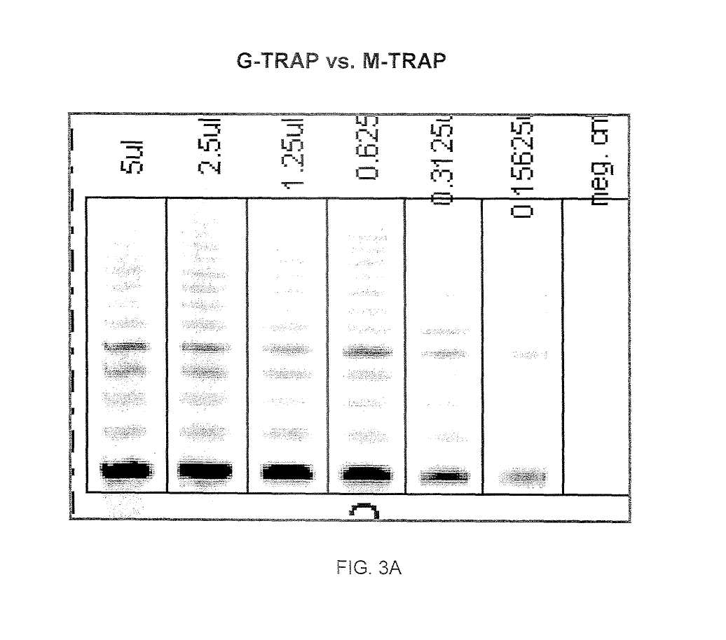

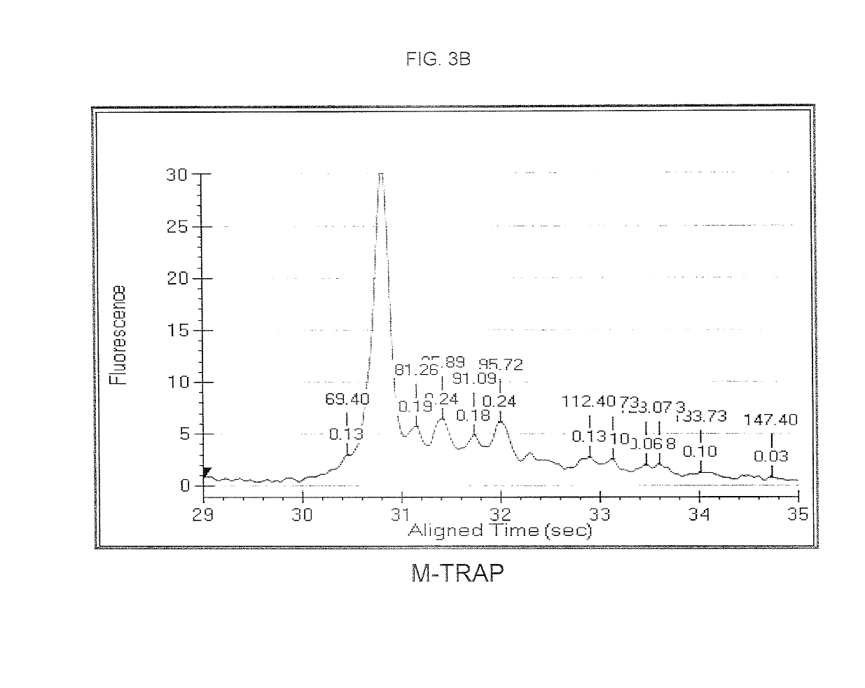

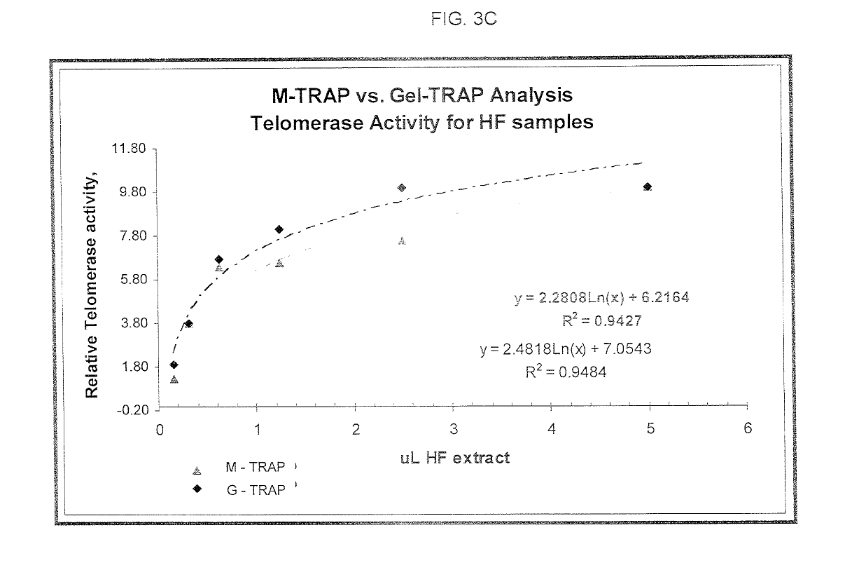

FIG. 3A is a picture of an acrylamide gel. FIG. 3B is the readout of the microfluidic device from the same TRAP assay. FIG. 3C is a comparison of the relative telomerase activity of the analysis of the TRAP reaction using the acrylamide gel versus the microfluidic device over a range of different concentrations of hair follicle extract with 33 cycles of PCR. For ease of comparing the data, curve fitting using a log transformation of the extract volume was conducted to generate slope and correlation data. 1/384 of hair follicle telomerase activity could be detected by the microfluidics device with 30 to 33 PCR cycles. 1/384 of hair follicle telomerase activity could be detected by acrylamide gel with 28 to 30 PCR cycles. It was found that the microfluidic device provided results that were comparable to the acrylamide gel analysis over the range of concentration of cell extracts. Similar results were seen when different numbers of PCR cycles were used. The microfluidic device provided higher throughput with smaller sample volume and digital data.

### Example 2

### Telomerase Activity Measurement in Imetelstat Treated Human Hair Follicle, A549 and HEK293 Cells

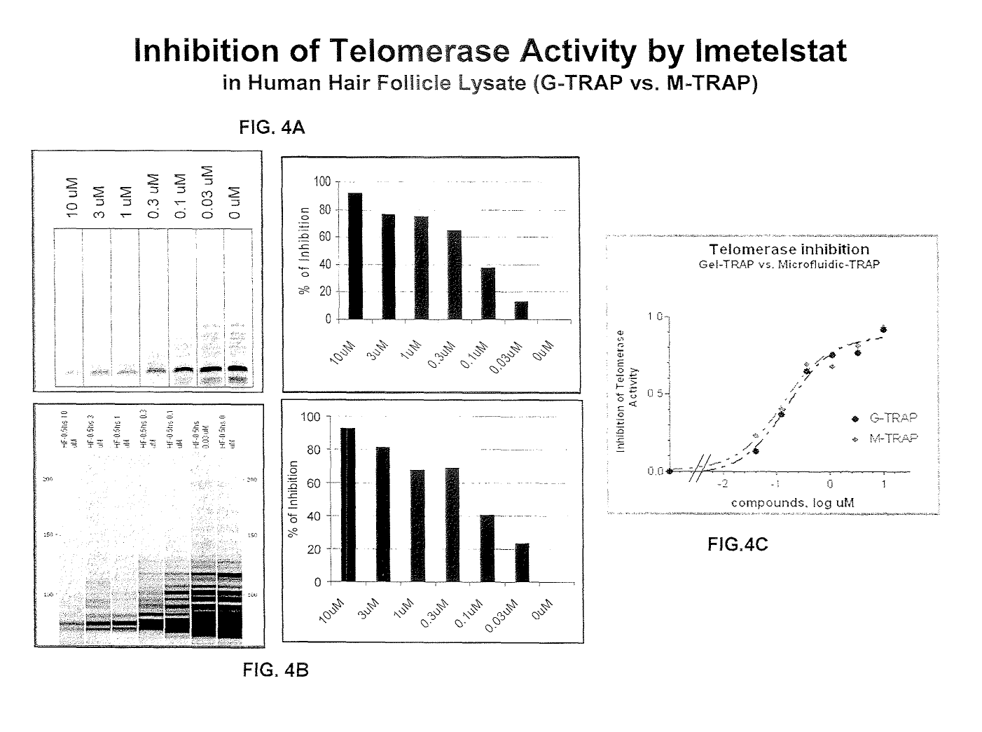

Imetelstat (GRN163L) is a telomerase inhibitor developed by Geron as an anti-cancer drug to suppress tumor cell growth. In order to measure the inhibition effect of Imetelstat in human hair follicle lysate, 15 μL of hair follicle lysate (¼ of a HF extract) from a pooled lysate or 15 μL of extract from 200 cells of A549 (non-small cell lung cancer) cells (ATCC, Maryland) or HEK293 (human embryonic kidney) cells (ATCC, Maryland) obtained by the method of Example 1 were treated with various amounts of Imetelstat. Different concentrations of Imetelstat were added to separate 15 μL volumes of A549 or HEK 293 cell extract or human hair follicle lysate. The range of Imetelstat was as indicated in FIGS. 4, 5 and 6. The hair follicle lysate was incubated on ice for four hours. The A549 or HEK 293 cell extract was incubated for one hour. 5 μL of each Imetelstat treated lysate or extract was used for the TRAP assay and measurement in the microfluidic device by the method in Example 1.

FIG. 4A is a picture of an acrylamide gel and bar graph and FIG. 4B is the readout of the microfluidic device and bar graph both from the same TRAP analysis after treatment of the hair follicle extract with different concentrations of Imetelstat (GRN163L). FIG. 4C is a graph showing the inhibition of telomerase activity with different concentrations of Imetelstat. It was found that the readout from the microfluidic device was comparable to the acrylamide gel readout.

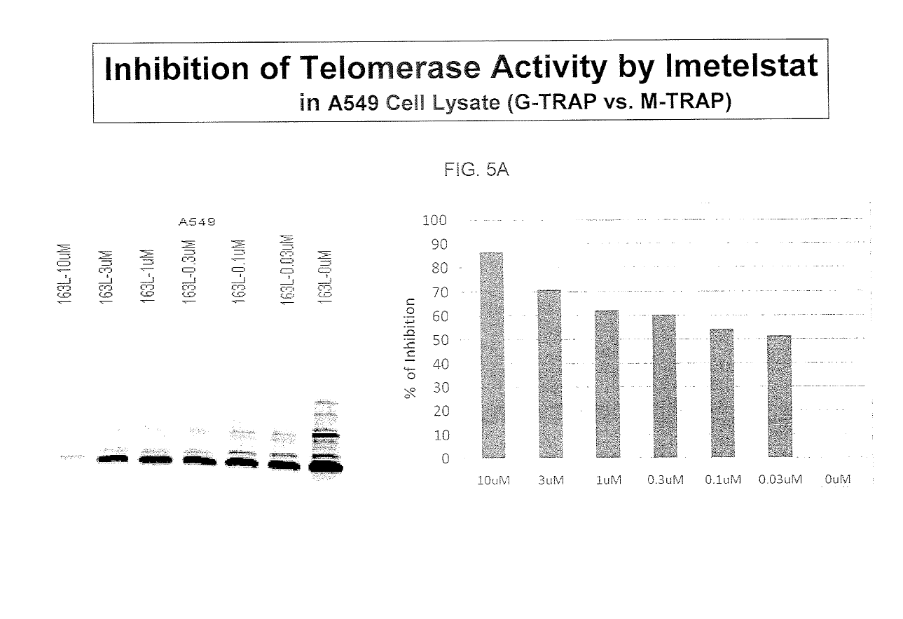

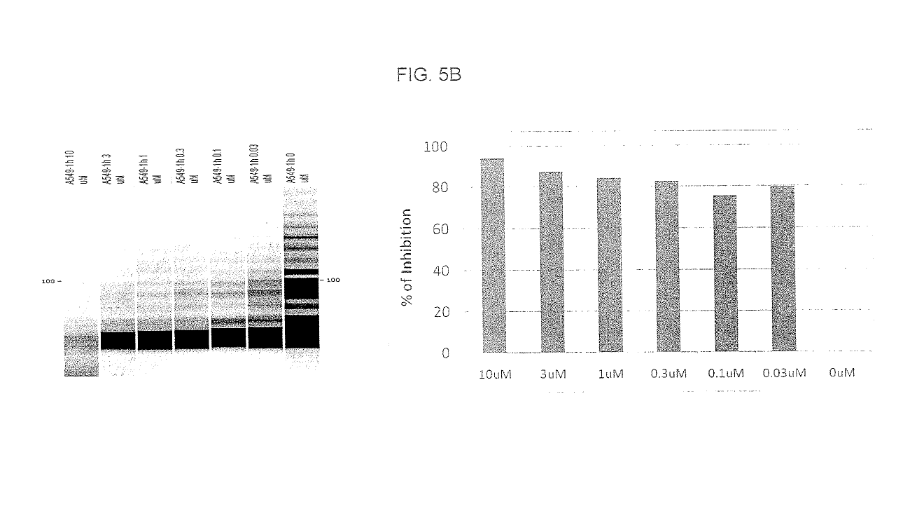

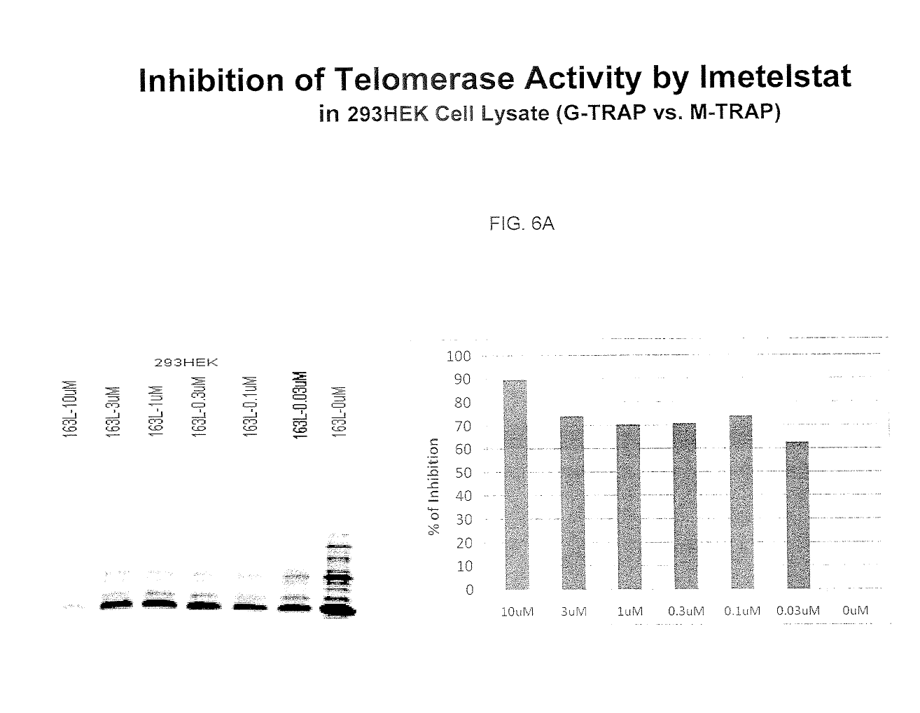

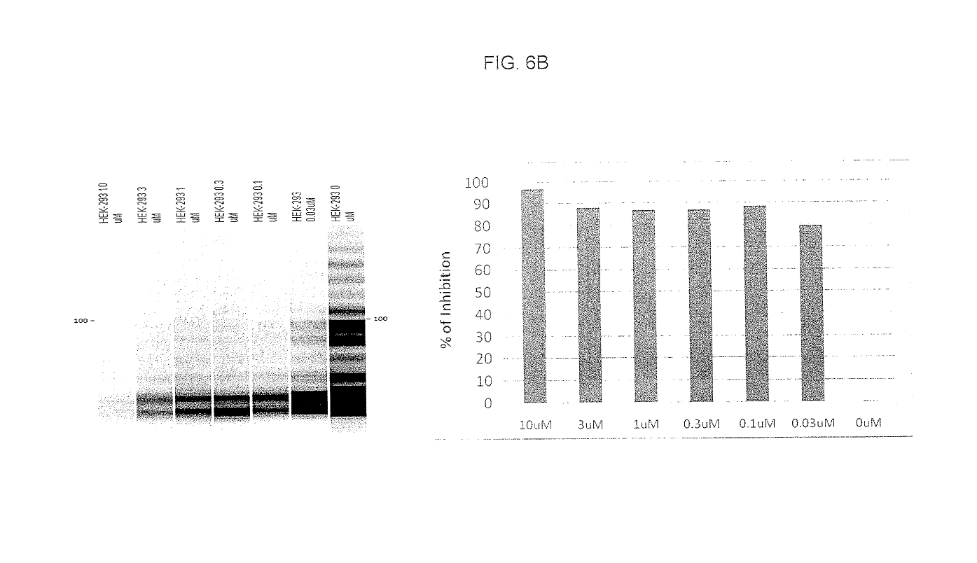

FIG. 5 shows the results for the A549 cell lysate and FIG. 6 shows the results for the HEK293 cell lysate. It was found that the readout from the microfluidic device was comparable to the acrylamide gel readout.

Although the invention has been described with respect to particular embodiments and applications, those skilled in the art will appreciate the range of applications and methods of the invention disclosed herein.

1. A method of measuring telomerase repeat amplification products in a cell or tissue reaction mixture comprising flowing the telomerase repeat amplification products in a channel of a microfluidic device and measuring the amount of telomerase repeat amplification products in the reaction mixture.

2. A method of measuring telomerase activity in a cell or tissue extract, the method comprising the steps of:
(a) placing an aliquot of cell or tissue extract in a reaction mixture comprising a telomerase substrate and a buffer in which telomerase can catalyze extension of said telomerase substrate by addition of telomeric repeat sequences to generate telomeric extension products which are then amplified to generate telomerase repeat amplification products;
(b) separating the telomerase repeat amplification products in a channel of a microfluidic device, and
(c) measuring the amount of telomerase repeat amplification products in the reaction mixture.

3. The method of claim 2 wherein the amplification step of the method comprises: adding to said reaction mixture a primer comprising a sequence sufficiently complementary to a telomeric repeat to hybridize specifically thereto under conditions such that if a telomeric extension product is present in said reaction mixture, said primer will hybridize to said telomeric extension product and extend to form a complementary copy of said telomeric extension product, thereby forming telomerase repeat amplification products.

4. The method of claim 2 further comprising correlating the presence of telomerase activity in said cell extract with the presence of telomerase repeat amplification products and absence of telomerase activity in said cell sample with absence of said telomerase repeat amplification products.

5. A method for evaluating the biological response of a subject exposed to a telomerase modulator comprising:
(a) obtaining a cell or tissue sample from the subject,
(b) generating a telomerase repeat amplification product reaction mixture from the subject's cell or tissue sample,
(c) separating the telomerase repeat amplification products in a channel of a microfluidic device, and
(d) measuring the amount of telomerase repeat amplification products in the reaction mixture.

6. A method for evaluating the biological response of a subject exposed to a telomerase modulator comprising the steps of:
(a) placing an aliquot of cell or tissue extract from the subject after exposure to the telomerase modulator in a reaction mixture comprising a telomerase substrate and a buffer in which telomerase can catalyze extension of said telomerase substrate by addition of telomeric repeat sequences to generate telomeric extension products which are then amplified to generate telomerase repeat amplification products;
(b) separating the telomerase repeat amplification products in a channel of a microfluidic device,
(c) measuring the amount of telomerase repeat amplification products in the reaction mixture, and
(d) correlating the level of telomerase activity in said cell extract with the level of said extended telomerase substrate.

7. The method of claim 6 wherein the amplification step comprises adding to said reaction mixture a primer comprising a sequence sufficiently complementary to a telomeric repeat to hybridize specifically thereto under conditions such that if a telomeric extension product is present in said reaction mixture, said primer will hybridize to said telomeric extension product and extend to form a complementary copy of said telomeric extension product, thereby forming telomerase repeat amplification products.

8. The method of claim 7 further comprising heating said reaction mixture to denature said telomerase repeat amplification products; and cooling said reaction mixture to a temperature at which complementary nucleic acids can hybridize and said primer can extend if extended telomerase substrates are present.

9. The method of claim 1 wherein the telomerase substrate is labeled.

10. The method of claim 1 wherein the primer is labeled.

11. The method of claim 10 wherein the label is selected from the group consisting of a radioactive molecule, a fluorescent molecule, a phosphorescent molecule, a ligand for a receptor, biotin, and avidin.

12. The method of claim 1 wherein the telomerase repeat amplification product is labeled with an intercalating dye selected from the group consisting of a radioactive molecule or a fluorescent molecule.

13. The method of claim 1 wherein the telomerase substrate lacking a telomeric repeat sequence is 5′-AATCCGTCGAGCAGAGTT-3′ (SEQ ID NO:1).

14. The method of claim 3 wherein the primer comprises a non-telomeric repeat sequence at a 5′-end of said primer.

15. The method of claim 14 wherein the primer is 5′-CCCTTACCCTTACCCTTACCCTAA-3′ (SEQ ID NO: 2), 5′-GCGCGGCTAACCCTAACCCTAACC-3′ (SEQ ID NO:3) or 5′-GCGCGGCTTACCCTTACCCTTACCCTAACC-3′ (SEQ ID NO:4).

16. The method of claim 1 wherein further comprising normalizing the level of telomerase activity in the cell extract relative to the amount of RNA or protein in the cell extract.

17. The method of claim 1 wherein the cell or tissue extract is obtained from a mammal.

18. The method of claim 17 wherein the cells are cancer cells, skin cells, hair follicle cells, or blood cells.

19. The method of claim 1 wherein the microfluidic device comprises a detector selected from the group comprising a spectrophotometer, a fluorescent detector, a fluorescence polarization detector or a scintillation counting device.

20. The method of claim 19 wherein the detector is coupled to a computer which computer comprises instructions that convert a signal from the detector into the amount of the telomerase repeat amplification products in the reaction mixture.
